(19) United States
(12) Patent Application Publication
(10) Pub. No.: US 2020/0268652 A1
(43) Pub. Date: Aug. 27, 2020

(54) CANNABIS INFUSED BUBBLE GUM FOR SUB-LINGUAL DELIVERY OF CANNABINOIDS

(71) Applicant: Betty Hess, Sumner, WA (US)

(72) Inventor: Betty Hess, Sumner, WA (US)

(21) Appl. No.: 16/874,034

(22) Filed: May 14, 2020

Related U.S. Application Data
(63) Continuation of application No. 15/155,072, filed on May 15, 2016.

(51) Int. Cl. A61K 9/68; A23G 4/06; A61K 31/047; A23G 4/20; A23G 4/10; A61K 36/185; A61K 31/352

(52) U.S. Cl. CPC A61K 9/0058; A23G 4/062; A61K 31/047; A23G 4/20; A23V 2002/00; A23G 4/068; A61K 36/185; A61K 31/352; A23G 4/10

(57) ABSTRACT

The invention relates to a product and process for sub-lingual delivery of cannabinoids. The product is a natural bubble gum much like old fashioned bubble gum. The sublingual delivery can aid in controlling dosage as well as the psychosomatic and physiological effects typical to a cannabis infused edible product.

### CROSS REFERENCE TO RELATED APPLICATIONS

This application is a continuation of U.S. application Ser. No. 15/155,072 filed on May 15, 2016, which is incorporated herein by reference in its entirety as though fully set forth herein.

### FIELD

The invention relates to a novel product and process for sub-lingual delivery of cannabinoids wherein a natural bubble gum is the vehicle for sub-lingual delivery of measured amounts of cannabinoids as produced by a specific process as disclosed herein.

### BACKGROUND OF THE INVENTION

The invention most closely corresponds with USPTO Class 424/48 wherein Class 424 relates to bio-effecting and body treatment drug compositions, and sub-class 48 which includes chewing gum types.

In its simplest form, the invention relates to a novel product and process for sub-lingual delivery of cannabinoids when a person or patient does not desire to or cannot swallow an “edible” as known in the medical or recreational cannabis industry.

The inventive product is a bubble gum which smells, tastes, and chews like an old-fashioned bubble gum such as “Dubble Bubble”™. The method for infusing the bubble gum is novel and comports to regulatory requirements that most states have in place for infused cannabis products. The bubble gum and method ensure that the product is non-GMO, gluten free, and can be embodied in a fully organic form with two minor ingredient modifications. Both embodiments will be discussed herein.

When you consume marijuana in an ingestible form, its THC is metabolized by the liver, which converts it to 11-hydroxy-THC. This active metabolite is particularly effective in crossing the blood-brain barrier, resulting in a more intense response. Inhaled THC undergoes a different metabolic process because rather than passing through the stomach and then the liver, the THC travels directly to the brain. Many people do not want to inhale THC or smoke in any form. The inventive product and process results in a safe, sub-lingual delivery of cannabinoids.

### THE INVENTION SUMMARY, OBJECTS AND ADVANTAGES

It is fairly well known that many inexperienced consumers have ingested too much THC via some edible product they purchased in a state where such infused edibles are legal. One major problem is the actual dose in a given edible. Consumers are not typically aware of what their threshold is for any given dose. When one consumes for example, a 25 mg edible, they consume the entire thing and all of the THC processes through a metabolic process similar to that of food. Results are not immediate, and oftentimes people will consume more since they “don't feel anything”.

The obvious problem with consuming a second or third edible item, is the time delay of the metabolic process. After feeling little effect after a first ingestion, the buildup of THC in a second or third edible can be a concern. Indeed, a THC “overdose” can occur, and the person may have a very negative experience, or even end up in an emergency room. Proper dosage and methods of production are crucial for the avoidance of negative effects due to dosing errors. The inventive bubble gum is strategic in avoiding the “overdose” effect because it is not swallowed as a whole, and thus the THC release is via a slower, sub-lingual delivery method.

When you take medicines by mouth, they move through the digestive tract and are taken up by internal organs like the stomach and small intestine. Often, they are then sent to the liver, where they might be chemically altered. Finally, they are released into the bloodstream. Just as it does with food, the body tries to chemically break down medicines as soon as they enter the body. Most drugs taken by mouth enter the stomach or small intestine and then are sent to the liver. The liver contains protein molecules called enzymes that chemically modify drugs and other non-food substances. The chemical alteration of a medicine by the body is called drug metabolism. Often, when a drug is metabolized by the body, it is converted into products called metabolites. Usually, these metabolites are not as strong as the original drug.

One advantage of a sub-lingual delivery is that the metabolization tends to occur a bit quicker than if one swallowed an infused fruit chew for example. Since the bubble gum releases medication through the saliva, it is converted fairly quickly, and in kind, a user should feel effects quickly and make an informed decision to discontinue or control further consumption.

Another advantage of the sub-lingual approach of the inventive bubble gum is that a user may also place the piece of gum aside and return to chew it further at a later time. Again, this serves in aiding the dose control. When the bubble gum seems to have lost its taste, a user simply throws it out.

A preferred embodiment utilizes corn syrup and confectioner's sugar. In a second embodiment, the bubble gum can be made to comport with national standards of fully organic products wherein the corn syrup and sugar are replaced by avocado oil and stevia (a natural sweetener). No further modifications are required to make the inventive bubble gum 100% organic.

The product and process may be further controlled by the amount and potency of medicine or hash oil that is blended into the ingredient mix. Some medical patients may be prescribed a 25 mg portion, and recreational users may only be allowed a 10 mg portion. The milligram range is easy to control in the inventive method of making the bubble gum. Doses are fairly accurate based upon the mathematical relation between the solid and liquid ingredients and the oil which is infused therein.

Unlike other infused gums in the prior art, the inventive bubble gum is not coated with a sucrose shield, nor is it hard or “crumbly” when chewed. Nearly all infused gum in the prior art has a less than pleasant taste and texture. The inventive bubble gum is very much like the experience of chewing a pink piece of bubble gum from your youth. Soft and sweet, and malleable. The inventive gum congeals nearly immediately upon chewing and stays together in a rubbery bond. The medicinal results are a mellow feeling of relaxation and pain relief without the psychosomatic feeling of being “too high” and experiencing related negative effects.

### BRIEF DESCRIPTION OF THE DRAWINGS

The invention is described in further detail by reference to one (1) drawing in the form of a flow chart sufficient in detail to describe the invention in which:

FIG. 1 details the process by which the sub-lingual delivery of cannabinoid bubble gum product is produced.

### DETAILED DESCRIPTION, INCLUDING BEST MODES OF CARRYING OUT THE INVENTION

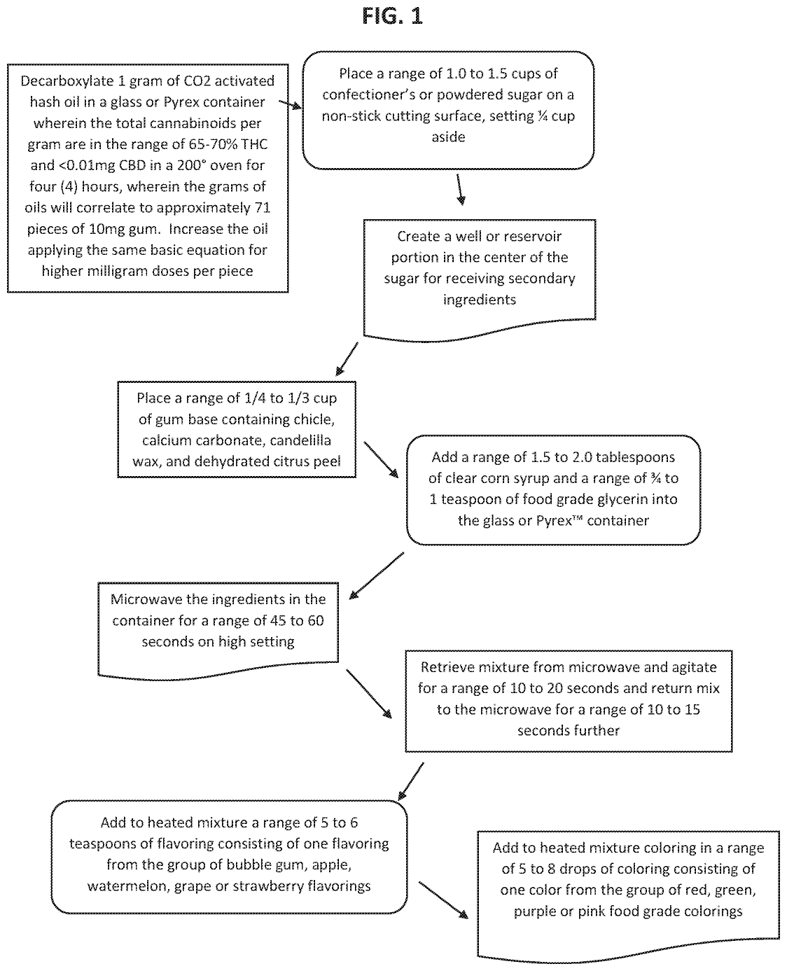

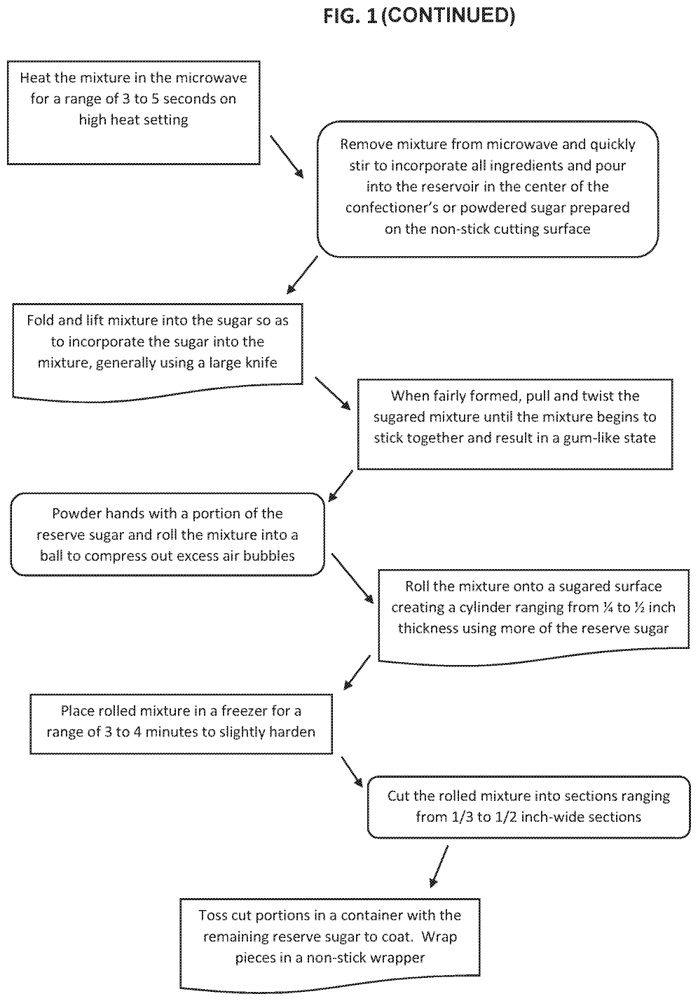

FIG. 1 is a flow chart of the product by process steps for producing the sub-lingual delivery of cannabinoids wherein the steps are as follows:

Decarboxylate 1 gram of CO2 activated hash oil in a glass or Pyrex™ container wherein the total cannabinoids per gram are in the range of 65-70% THC and <0.01 mg CBD in a 200° oven for four (4) hours;

Place a range of 1.0 to 1.5 cups of confectioner's or powdered sugar on a non-stick cutting surface, setting ¼ cup aside;

Create a well or reservoir portion in the center of the sugar for receiving secondary ingredients;

Place a range of ¼ to ⅓ cup of gum base in a large glass or Pyrex container; Add a range of 1.5 to 2.0 tablespoons of clear corn syrup and a range of ¾ to 1 teaspoon of food grade glycerin into the glass or Pyrex container;

Microwave the ingredients in the container for a range of 45 to 60 seconds on high setting;

Retrieve mixture from microwave and agitate for a range of 10 to 20 seconds and return

mix to the microwave for a range of 10 to 15 seconds further;

Add to heated mixture a range of 5 to 6 teaspoons of flavoring consisting of one flavoring from the group of bubble gum, apple, watermelon, grape or strawberry flavorings;

Add to heated mixture coloring in a range of 5 to 8 drops of coloring consisting of one color from the group of red, green, purple or pink food grade colorings;

Heat the mixture in the microwave for a range of 3 to 5 seconds on high heat setting;

Remove mixture from microwave and quickly stir to incorporate all ingredients and pour into the reservoir in the center of the confectioner's or powdered sugar prepared on the non-stick cutting surface;

Fold and lift mixture into the sugar so as to incorporate the sugar into the mixture, generally using a large knife;

When fairly formed, pull and twist the sugared mixture until the mixture begins to stick together and result in a gum-like state;

Powder hands with a portion of the reserve sugar and roll the mixture into a ball to compress out excess air bubbles;

Roll the mixture onto a sugared surface creating a cylinder ranging from ¼ to ½ inch thickness using more of the reserve sugar;

Place rolled mixture in a freezer for a range of 3 to 4 minutes to slightly harden; Cut the rolled mixture into sections ranging from ⅓ to ½ inch wide sections;

Toss cut portions in a container with the remaining reserve sugar to coat. Wrap pieces in a non-stick wrapper.

*Dubble Bubble and Pyrex are trademarked products of Fleer and Pyrex respectively.

1) A process to produce a product for sub-lingual delivery of cannabinoids, wherein the product is bubble gum and the process comprises the steps of;
a) Decarboxylating 1 gram of CO2 activated hash oil wherein the total cannabinoids per gram are 65-70% tetrahydrocannabinol (THC) and <0.01 mg cannabidiol (CBD) in an oven;
b) Placing 1.0 to 1.5 cups of sugar on a non-stick surface;
c) Creating a well or reservoir portion in the center of the sugar for receiving secondary ingredients;
d) Placing ¼ to ⅓ cup of a mixture comprising chicle, calcium carbonate, candelilla wax, and dehydrated citrus peel in a container;
e) Adding 1.5 to 2.0 tablespoons of clear corn syrup and ¾ to 1 teaspoon of food grade glycerin into the container;
f) Microwaving ingredients in the container for 45 to 60 seconds;
g) Agitating the ingredients in the container for 10 to 20 seconds and microwaving the ingredients in the container for another 10 to 15 seconds to obtain a heated mixture;
h) Adding 5 to 6 teaspoons of flavoring to the heated mixture, wherein the flavoring is selected from the group consisting of bubble gum, apple, watermelon, grape and strawberry;
i) Adding coloring in a range of 5 to 8 drops of coloring to the heated mixture, wherein the coloring is selected form the group consisting of red, green, purple and pink;
j) Re-heating the heated mixture in a microwave for a range of 3 to 5 seconds on high heat setting;
k) Removing the heated mixture from microwave, stirring to incorporate all ingredients and pouring into the well or reservoir portion in the center of the sugar;
l) Folding and lifting mixture into the sugar so as to incorporate the sugar into the heated mixture, wherein a sugared mixture is produced;
m) Pulling and twisting the sugared mixture until the sugared mixture begins to stick together and results in a gum-like state;
n) Rolling the sugared mixture into a ball to compress out excess air bubbles;
o) Rolling the sugared mixture onto a sugared surface creating a cylinder ranging from ¼ to ½ inch thickness;
p) Placing rolled mixture in a freezer for a range of 3 to 4 minutes to obtain a hardened rolled mixture;
q) Cutting the hardened rolled mixture into sections ranging from ⅓ to ½ inch-wide sections to obtain cut portions;
r) Tossing the cut portions in sugar to coat; and
s) Wrapping the cut portions in a non-stick wrapper.

2) The process for sub-lingual delivery of cannabinoids of claim 1, wherein the corn syrup is replaced with avocado oil and stevia.

3) A product produced by the process of claim 1.
